(19) United States
(12) Patent Application Publication
(10) Pub. No.: US 2022/0370233 A1
(43) Pub. Date: Nov. 24, 2022

(54) Barrier Press

(71) Applicant: Keith Lawton, Gillette, WY (US)

(72) Inventor: Keith Lawton, Gillette, WY (US)

(21) Appl. No.: 17/323,953

(22) Filed: May 18, 2021

(51) Int. Cl. A61F 5/448; A61F 5/449

(52) U.S. Cl. CPC A61F 5/448; A61F 5/449

(57) ABSTRACT

A barrier press is presented that comprises an external plate attached to a barrel tube that has a bag slot for the insertion of a body fluid bag that attaches to a stoma hole via the skin barrier, the barrel tube has a pressure ring to keep the skin barrier attached to the area around the stoma and in other embodiments an inner plate held in place by the pressure ring further reinforces the bag attachment to a body fluid bag user.

### FIELD OF THE INVENTION

The present invention relates to further securing and attaching a body fluid bag to a user that has a stoma for the release of bodily fluids.

### BACKGROUND OF THE INVENTION

After disease or injury, some people are forced to remove a body organ or have malfunctioning body organs. As a result of such injury or disease the person is required to proceed through their life with a stoma, or opening in their skin, which allows for body fluid to exit from the body organ to a collection bag through the stoma. In one example, a stoma is an opening on the abdomen that can be connected to the urinary system to allow urine to be diverted out of the body into a body fluid bag for collection and later disposal.

Typically, a body fluid bag is “worn” by the user by means of an adhesive outer skin barrier attached to the body fluid bag that sticks to the user's stomach, i.e., a skin barrier. There is a hole in the flange ring that surrounds the stoma. The adhesive is meant to prevent leaks, but often does not. A recent medical manufacturers report states that 85% of ostomy/stoma patients experience leaking, so this is a well-known issue in the medical arts. Barrier manufacturers' suggested wear times for the current barriers on the marketplace today range from changing the barrier daily to 5-7 days maximum due to the leaking issue. Multiple replacements are costly. Accompanying the inconvenience of the leaks, body fluid bag users also suffer from infected skin issues and the general embarrassment of a urine or other bodily fluid leaking onto clothes. Thus, there is a long felt need to create an efficient, reliable and cleaner method to contain and secure the body fluid bag to a stoma and thereby help users to save money, increase their activity level and have confidence that they will remain leak-free.

### SUMMARY OF THE INVENTION

Accordingly, it is an object of embodiments of the present invention to provide for a barrier press to secure a body fluid bag to a user. Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims. To achieve the foregoing and other objects, and in accordance with the purposes of the present invention, as embodied and broadly described herein, the invention comprises a barrier press having an outer plate and a barrel tube, the outer plate connected to the barrel tube; the outer plate further having a first stabilizer wing, a second stabilizer wing opposing the first stabilizer wing, and a port ring disposed between the first stabilizer wing and the second stabilizer wing; the barrel tube further having a distal end and a proximal end, the proximal end having pressure ring and a bag slot, the bag slot disposed between the pressure ring and the distal end of the barrel tube, the distal end of the barrel tube in contact with the port ring of the outer plate.

Benefits and advantages of the present invention include, but are not limited to providing a means to affix a body fluid bag to a user having a stoma by means of a ring adding pressure to the adhesive and against the user.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a top side perspective view of one embodiment of the outer plate of the barrier press invention.

FIG. 2 illustrates another top and side perspective view of another embodiment of the outer plate of the instant barrier press invention.

FIG. 3 illustrates a top and side perspective view one embodiment of the outer plate of the instant invention.

FIG. 4 illustrates a top and side rear perspective view of one embodiment of the barrel tube of the instant invention.

FIG. 5 illustrates a side and top rear perspective view of one embodiment of the barrel tube and outer plate instant invention prior to or after connection.

FIG. 6 illustrates a side and top perspective view of one embodiment of the instant invention depicting of one embodiment of the barrel tube and outer plate instant invention where the two elements are in contact with one another.

FIG. 7 illustrates a top perspective view of one embodiment of the inner plate instant invention.

FIG. 8 illustrates a top perspective view of one embodiment of the instant invention wherein the inner plate is about to the barrel tube.

FIG. 9 illustrates an inner top perspective view of one embodiment of the instant invention depicting the inner plate contacting the pressure tube and the outer plate.

FIG. 10 illustrates an outer top perspective view of one embodiment of the instant invention depicting the inner plate contacting the pressure tube and the outer plate.

FIG. 11 illustrates a side view of one embodiment of the instant invention depicting the inner plate contacting the pressure tube and the outer plate.

FIG. 12 illustrates another embodiment of the instant invention depicting the attachment of the body fluid bag.

FIG. 13 illustrates another embodiment of the instant invention depicting the attachment of the body fluid bag.

FIG. 14 illustrates another embodiment of the instant invention depicting the attached of the body fluid bag.

FIG. 15 illustrates an inner side perspective view of one embodiment of the instant invention depicting the attached of the body fluid bag to one embodiment of the instant invention having the inner plate.

FIG. 16 illustrates a side perspective view another embodiment illustrating the attached of the body fluid bag.

FIG. 17 illustrates an inner side perspective view of one embodiment of the instant invention depicting the attached body fluid bag.

FIG. 18 illustrates an outer view of one embodiment of the instant invention depicting the body fluid bag attached to the one embodiment of the invention attached to an end user.

### DETAILED DESCRIPTION OF THE INVENTION AND DRAWINGS

Reference will now be made in detail to embodiments of the invention, examples of which are illustrated in the accompanying drawings. Throughout the following detailed description, the same reference characters refer to the same or similar elements in all Figures. In this detailed description, reference is to a few embodiments of the instant invention, as illustrated in the accompanying and above-referenced drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention.

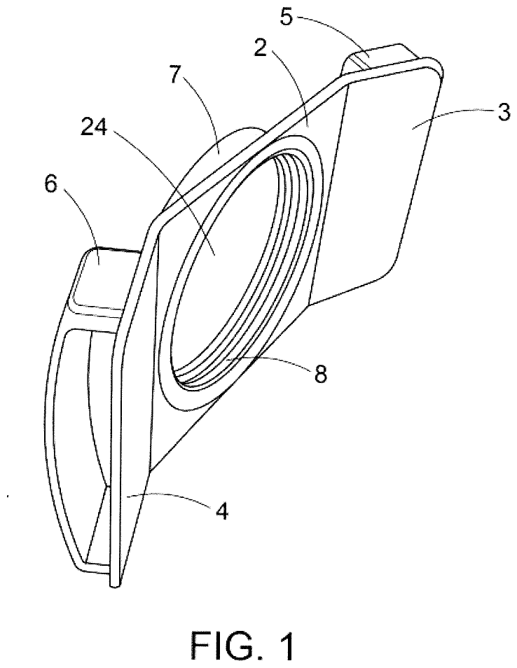

FIG. 1 depicts one embodiment of the instant invention, a barrier press outer plate 2, adapted for connection to a barrel tube (not depicted). The outer plate 2 is attached to a first stabilizer wing 3 and the outer plate 2 is attached to a second stabilizer wing 4, which opposes the first stabilizer wing 3 on the opposite side of the barrier plate 2. A port ring 7 is disposed between the first stabilizer wing 3 and the second stabilizer wing 4. The outer plate further comprises a first belt loop 5 attached to the first stabilizer wing 3 and a second belt loop 6 attached to the second stabilizer wing 4. The port ring 7 further comprises a threaded internal surface 8. In many embodiments, the not depicted barrel tube has a threaded external surface that engages the threaded internal surface 8 of the port ring 7. FIG. 1 further depicts a view port 24 within the port ring 7.

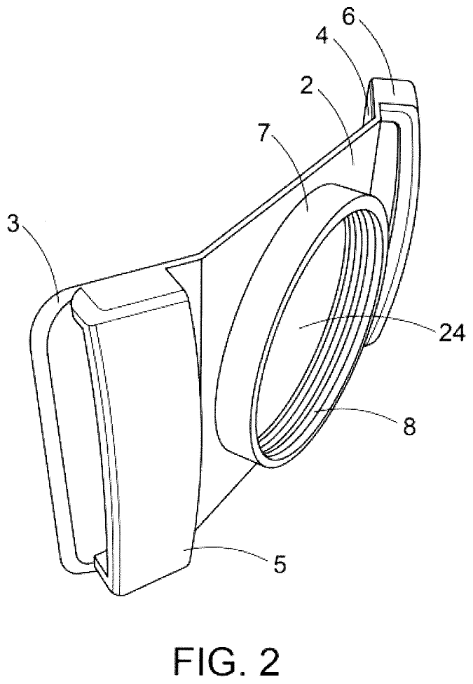

Similar to FIG. 1, FIG. 2 depicts one embodiment of the barrier press outer plate 2. The outer plate 2 is attached to a first stabilizer wing 3 and the outer plate 2 is attached to a second stabilizer wing 4, which opposes the first stabilizer wing 3 on the opposite side of the barrier plate 2 The port ring 7 is disposed between the first stabilizer wing 3 and the second stabilizer wing 4. The outer plate further comprises the first belt loop 5 attached to the first stabilizer wing 3 and the second belt loop 6 attached to the second stabilizer wing 4. The port ring 7 further comprises the threaded internal surface 8. FIG. 2 further depicts the view port 24 within the port ring 7.

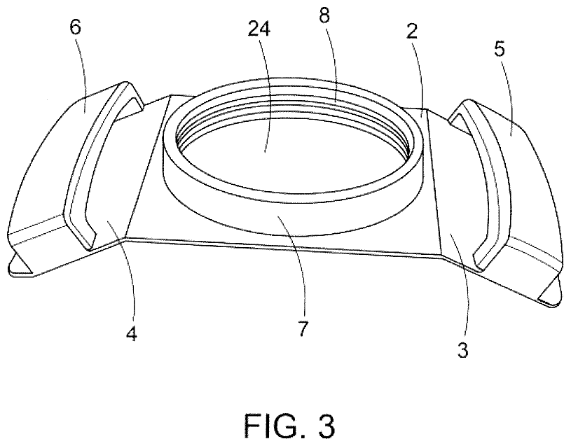

Similar to FIG. 1 and FIG. 2, FIG. 3 depicts one embodiment of the barrier press outer plate 2. The outer plate 2 is attached to a first stabilizer wing 3 and the outer plate 2 is attached to a second stabilizer wing 4, which opposes the first stabilizer wing 3 on the opposite side of the barrier plate 2. The port ring 7 is disposed between the first stabilizer wing 3 and the second stabilizer wing 4. The outer plate further comprises the first belt loop 5 attached to the first stabilizer wing 3 and the second belt loop 6 attached to the second stabilizer wing 4. The port ring 7 further comprises the threaded internal surface 8. FIG. 3 further depicts the view port 24 within the port ring 7.

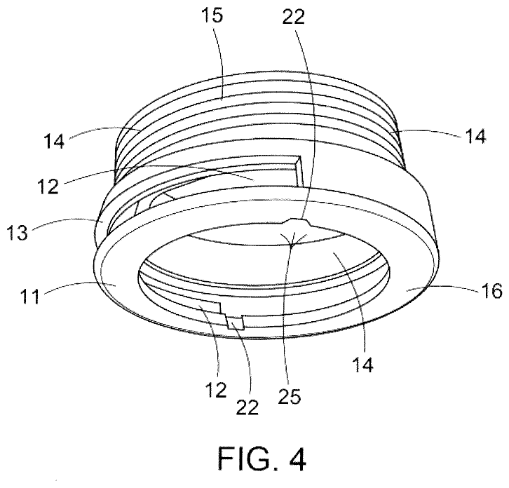

FIG. 4 depicts the barrel tube 13 having a distal end 14 and a proximal end 11. The proximal end 11 has a pressure ring 16 and a bag slot 12. The bag slot 12 is disposed between the pressure ring 16 and the distal end 11 of the barrel tube 13. The bag slot 12 partially surrounds the barrel tube 13. The distal end 14 of the barrel tube 13 adapted to contact the port ring of the outer plate (not depicted in FIG. 4). The distal end 14 of the barrel tube 13 further comprises a threaded external surface as depicted in FIG. 4. In this embodiment, the pressure ring 16 has a flattened surface. The pressure ring 16 comprises at least one notch 22. In the embodiment depicted in FIG. 4, the pressure ring 16 further comprises an orifice 25 that extends at least as far into the barrel tube as the bag slot 12. In actual use, a collection bag slides through the bag slot 12 and bag's flange collection ring insert through the orifice 25 of the pressure ring 16. In this embodiment as depicted in FIG. 4, the distal end 14 comprises a threaded external surface 15. The threaded external surface 15 is adapted to engage the threaded internal surface of the port ring of the barrier plate (not depicted in FIG. 4).

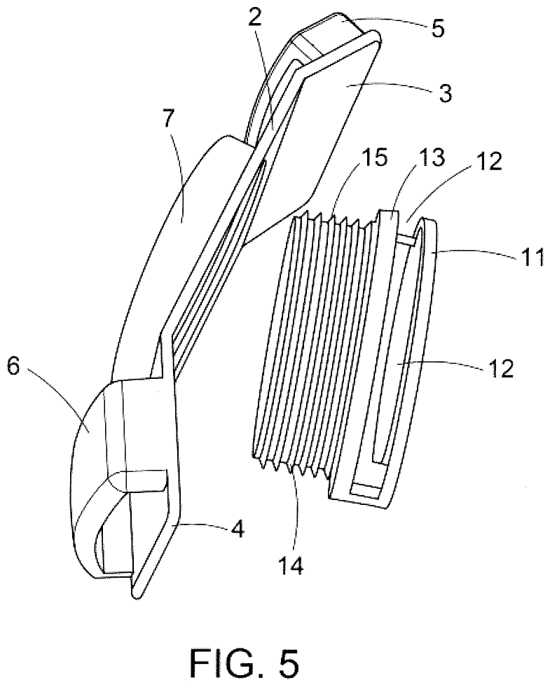

FIG. 5 depicts the outer plate 2 and the barrel tube 13 prior to connection or after connection. The threaded external surface 15 of the distal end 14 of the barrel tube 13 will connect to the threaded internal surface of the port ring 7 of the outer plate 2. The bag slot 12 is disposed between the proximal end 11 and the distal end 14.

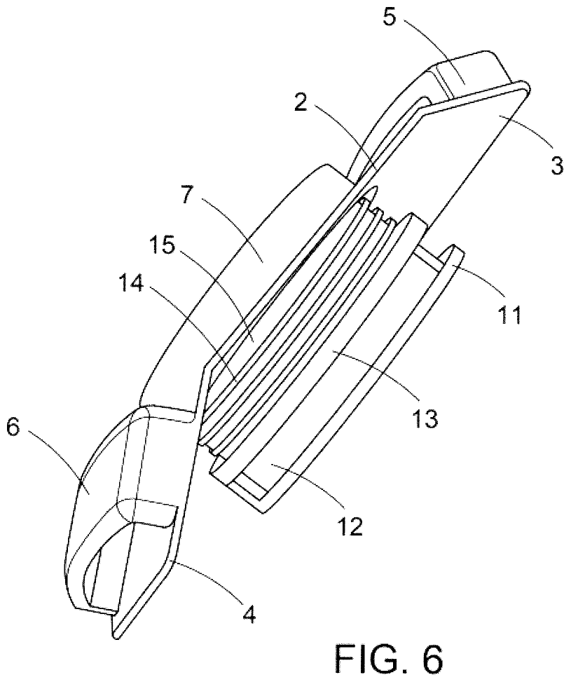

FIG. 6 depicts connection of the outer plate 2 and the barrel tube 13. The threaded external surface 15 of the distal end 14 of the barrel tube 13 is depicted as connected to the threaded internal surface of the port ring 7 of the outer plate 2. The bag slot 12 is disposed between the proximal end 11 and the distal end 14.

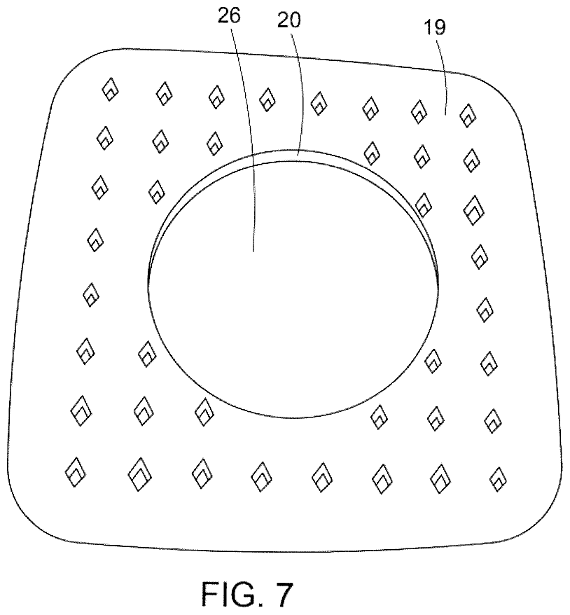

FIG. 7 depicts an inner plate 19 adapted to contact the pressure ring (not depicted). The inner plate has an outer circumference and an inner circumference 20. The inner circumference surrounds a void space 26.

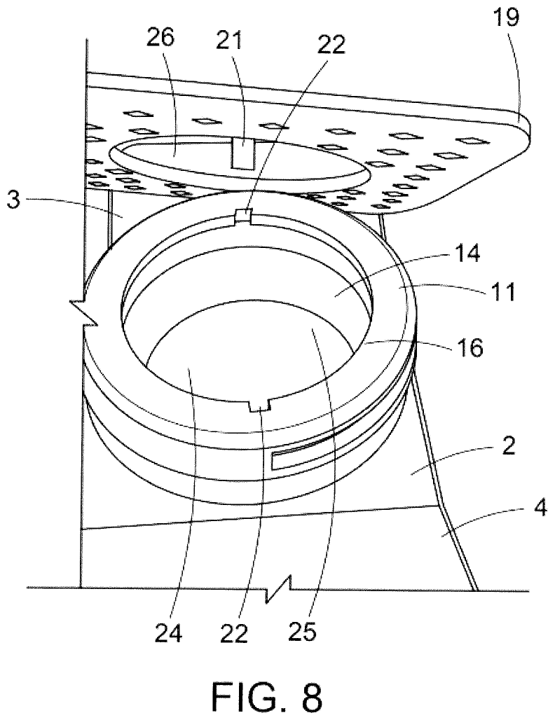

FIG. 8 depicts the inner plate 19 initially contacting the pressure ring 16 of the proximal end 11 of the barrel tube. The inner plate has at least one tab 21 disposed along the inner circumference of the inner plate, which engages the notch 22 of the pressure ring 16. The void space 26 of the inner plate 19 aligns with the orifice 25 of the barrel tube. In FIG. 8, the barrel tube is attached to the outer plate 2.

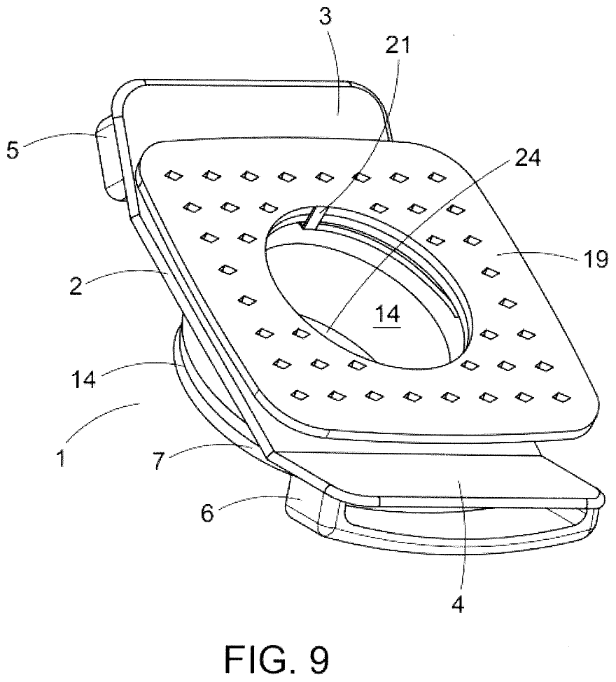

FIG. 9 depicts the barrier press 1 wherein the inner plate 19 is connected to the barrel tube which is connected to the outer plate 2. The inner plate 19 attaches to a back side of an adhesive pad of a surrounding adhesive cloth. The adhesive cloth encircles the adhesive ring that surrounds a skin barrier that adheres to the skin of the user. The body fluid bag attaches to the skin barrier via a flange.

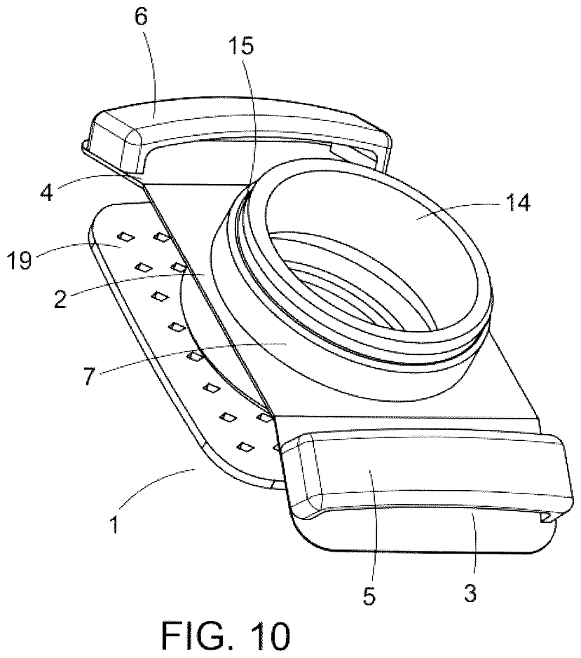

FIG. 10 depicts the barrier press 1 wherein the outer plate 2 has received the barrel tube. The distal end 14 of the barrel tube has extended into and through the view port of the outer plate 2. The inner plate 19 is attached to the barrel tube.

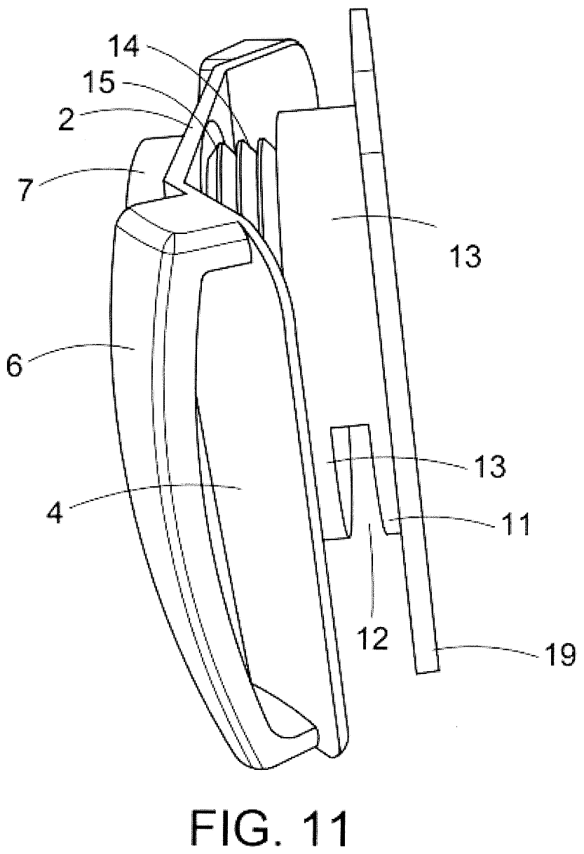

FIG. 11 depicts a side view of the barrier press as depicted in FIG. 9 and FIG. 10. The distal end 14 of the barrel tube has contacted the port ring 7 and the barrel tube has extended into the view port of the external plate 2. The inner plate 19 is attached to the barrel tube.

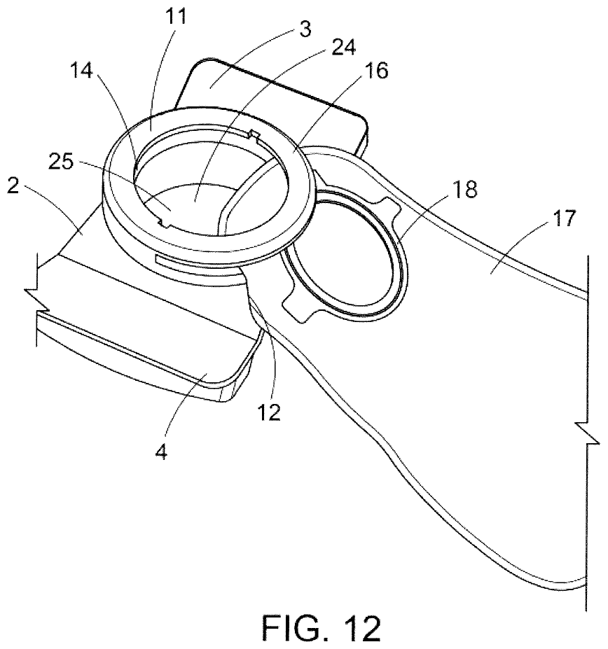

FIG. 12 depicts the insertion of the body fluid bag 17 though the bag slot of the barrel tube. The body fluid bag 17 has a flange 18, which will extend over the top of the pressure ring 16 of the proximal end 11 of the barrel tube.

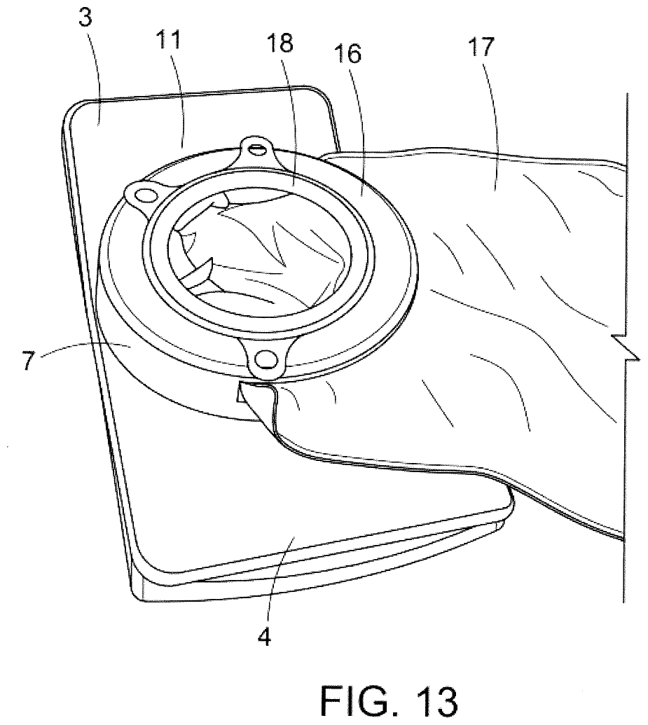

FIG. 13 depicts the complete insertion of the body fluid bag 17 though the bag slot of the barrel tube. The flange 18 of the body fluid bag 17 extends over the top of the pressure ring 16 of the proximal end 11 of the barrel tube.

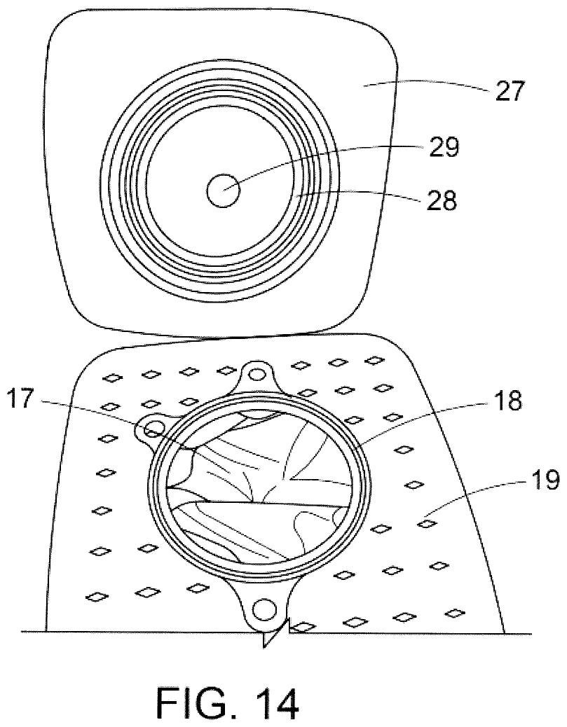

FIG. 14 depicts the flange 18 of the body fluid bag 17 that will be attached to the skin barrier 27 at the point of the skin barrier flange 28. The skin barrier 27 has a stoma hole 29 to be placed over a stoma of the user. In some embodiments of the instant invention and as shown in FIG. 14, the inner plate 19 makes contact with the supporting skin barrier surrounding cloth to further secure the skin barrier to the user. In other embodiments, the inner plate 19 is not used, and the pressure is applied to only the skin barrier portion between the stoma hole 29 and the skin barrier flange 28.

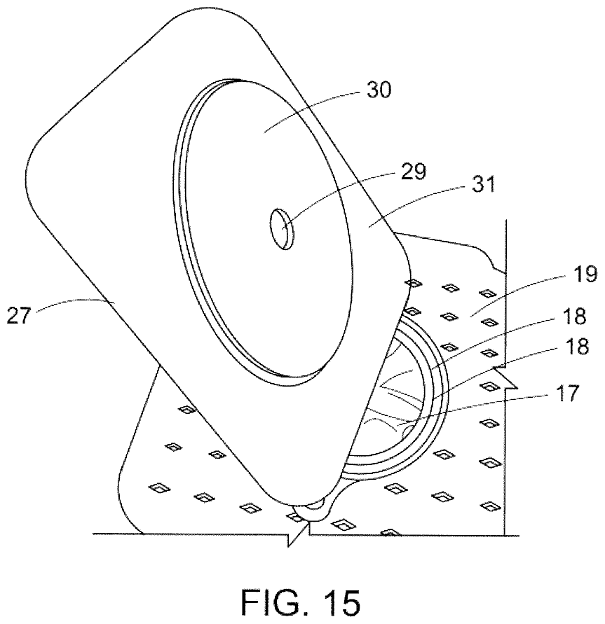

FIG. 15 depicts the adhesive side of the skin barrier 27 that will attach to the user's skin. FIG. 15 depicts the stoma hole 29 and the adhesive skin barrier portion 30. The skin barrier portion 30 is disposed between the indent of the skin barrier flange and the stoma hole 29. In the embodiment depicted in FIG. 15, the additional adhesive outer skin outer skin support cloth 31 would make contact with the user on the user skin. The inner plate 19 would provide additional pressure to maintain the adhesion to the user and prevent leakage of the body fluid bag (not depicted). Without the instant invention barrier press, the adhesive side of the skin barrier quickly weakens over time though erosion of the adhesive that causes increased leaking. The problematic leaking of the skin barrier attachment is stopped through the use of the instant invention barrier press.

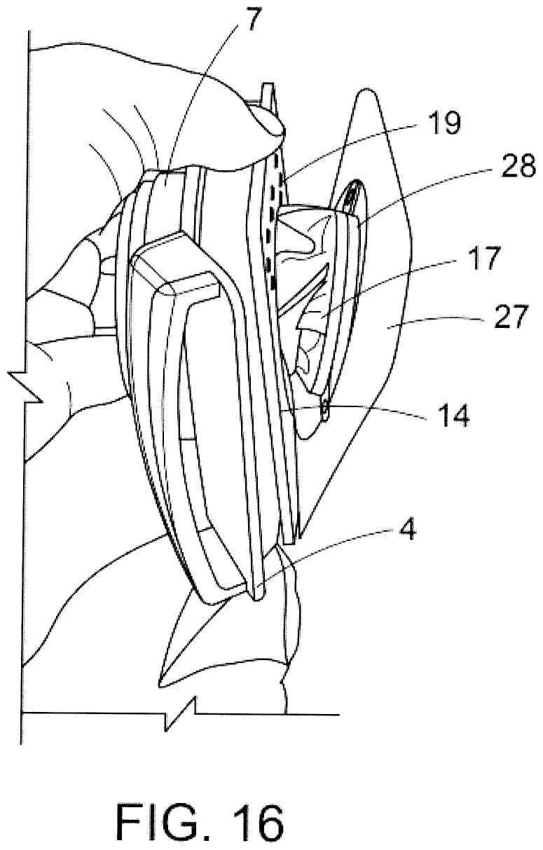

FIG. 16 depicts the body fluid bag 17, having the skin barrier 27 attached via the skin barrier flange 28. Although not depicted, the stoma hole would be placed over the user's stoma.

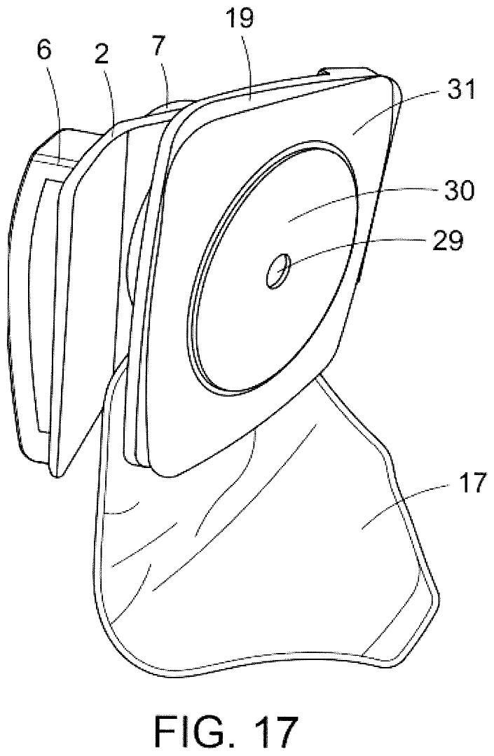

FIG. 17 depicts one embodiment of the instant invention wherein the adhesive outer skin barrier 31 and the adhesive skin barrier portion 30 are disposed between the indent of the skin barrier flange and the stoma hole 29. Again, in other embodiments, the outer skin barrier 31 and inner plate 19 are not employed and the pressurized contact is provided through the pressure ring.

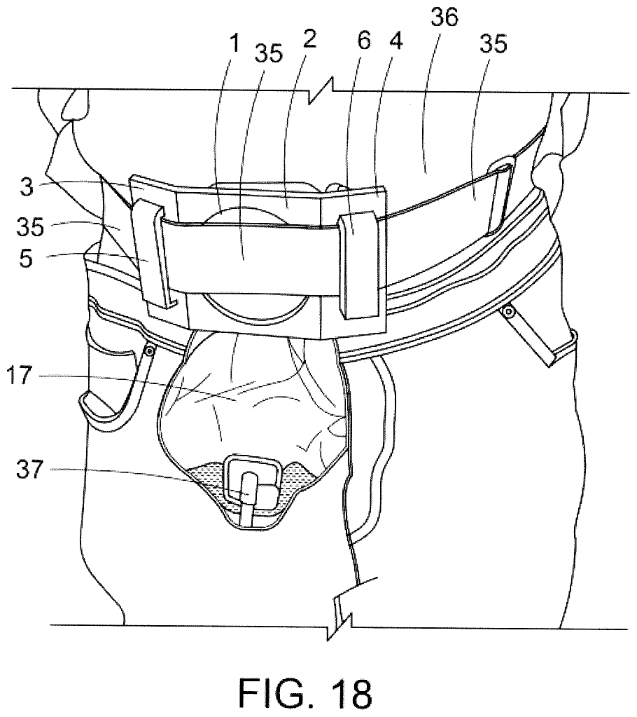

FIG. 18 depicts the body fluid bag 17 in pressurized contact via the barrier press in attachment to the end-user. A belt 35 is going through the first belt loop 5 and second belt loop 6 of the outer plate 2 of the barrier press. The body fluid bag 17 has a bag pouch valve 37 to allow for easy drainage.

The terms “certain embodiments”, “an embodiment”, “embodiment”, “embodiments”, “the embodiment”, “the embodiments”, “one or more embodiments”, “some embodiments”, and “one embodiment” mean one or more (but not all) embodiments unless expressly specified otherwise. The terms “including”, “comprising”, “having” and variations thereof mean “including but not limited to”, unless expressly specified otherwise. The enumerated listing of items does not imply that any or all of the items are mutually exclusive, unless expressly specified otherwise. The terms “a”, “an” and “the” mean “one or more”, unless expressly specified otherwise.

While this invention has been described in terms of several embodiments, there are alterations, permutations, and equivalents, which fall within the scope of this invention. Furthermore, unless explicitly stated, any method embodiments described herein are not constrained to a particular order or sequence. Further, the Abstract is provided herein for convenience and should not be employed to construe or limit the overall invention, which is expressed in the claims. It is therefore intended that the following appended claims be interpreted as including all such alterations, permutations, and equivalents as fall within the true spirit and scope of the present invention.

What is claimed is:

1. A barrier press comprising an outer plate and a barrel tube, said outer plate connected to said barrel tube;
said outer plate further comprising a first stabilizer wing, a second stabilizer wing opposing said first stabilizer wing, and a port ring disposed between said first stabilizer wing and said second stabilizer wing;
said barrel tube further comprising a distal end and a proximal end, said proximal end comprising pressure ring and a bag slot, said bag slot disposed between said pressure ring and said distal end of said barrel tube, said distal end of said barrel tube in contact with said port ring of said outer plate.

2. The barrier press of claim 1 wherein the outer plate further comprises a first belt loop attached to the first stabilizer wing.

3. The barrier press of claim 1 wherein the outer plate further comprises a second belt loop attached to the second stabilizer wing.

4. The barrier press of claim 1 wherein the port ring further comprises a threaded internal surface.

5. The barrier press of claim 1 wherein said distal end of said barrel tube further comprises a threaded external surface.

6. The barrier press of claim 1 wherein said pressure ring has a flattened face.

7. The barrier press of claim 1 wherein said pressure ring further comprises at least one notch.

8. The barrier press of claim 1 further comprising an inner plate in contact with said flattened face of said pressure ring.

9. The barrier press of claim 8, wherein said inner plate has an outer circumference and an inner circumference.

10. The barrier press of claim 9, wherein said inner plate has at least one tab disposed along the inner circumference of the inner plate.

11. A barrier press comprising an outer plate and a barrel tube, said outer plate connected to said barrel tube;
said outer plate further comprising a first stabilizer wing and a first belt loop attached to said first stabilizer wing, a second stabilizer wing opposing and a second belt loop attached to said second stabilizer wing, said first stabilizer wing, and a port ring disposed between said first stabilizer wing and said second stabilizer wing;
said barrel tube further comprising a distal end and a proximal end, said proximal end comprising pressure ring and a bag slot, said bag slot disposed between said pressure ring and said distal end of said barrel tube, said distal end of said barrel tube in contact with said port ring of said outer plate, said pressure ring further comprising a flattened face.

12. The barrier press of claim 11 wherein the port ring further comprises a threaded internal surface.

13. The barrier press of claim 11 wherein said distal end of said barrel tube further comprises a threaded external surface.

14. The barrier press of claim 1 wherein said pressure ring further comprises at least one notch.

15. The barrier press of claim 11 further comprising an inner plate in contact with said flattened face of said pressure ring.

16. The barrier press of claim 15, wherein said inner plate has an outer circumference and an inner circumference.

17. The barrier press of claim 15, wherein said inner plate has at least one tab disposed along the inner circumference of the inner plate.

18. A barrier press comprising an outer plate and a barrel tube, said outer plate connected to said barrel tube;
said outer plate further comprising a first stabilizer wing and a first belt loop attached to said first stabilizer wing, a second stabilizer wing opposing and a second belt loop attached to said second stabilizer wing, said first stabilizer wing, and a port ring disposed between said first stabilizer wing and said second stabilizer wing, and the port ring further comprises a threaded internal surface;
said barrel tube further comprising a distal end and a proximal end, said proximal end comprising pressure ring and a bag slot, said bag slot disposed between said pressure ring and said distal end of said barrel tube, said distal end of said barrel tube in contact with said port ring of said outer plate, said pressure ring further comprising a flattened face, said distal end of said barrel tube further comprises a threaded external surface.

19. The barrier press of claim 18 further comprising an inner plate in contact with said flattened face of said pressure ring.

20. The barrier press of claim 15, wherein said inner plate has an outer circumference and an inner circumference.
